(19) United States
(12) Patent Application Publication
(10) Pub. No.: US 2024/0207585 A1
(43) Pub. Date: Jun. 27, 2024

(54) CATHETER-LIKE DEVICE FOR SUPPLYING AND/OR DRAINING SUBSTANCES TO OR FROM THE BODY OF A PATIENT, AND METHOD FOR PRODUCING SUCH A DEVICE

(71) Applicant: Advanced Medical Balloons GmbH, Waghäusel (DE)

(72) Inventor: Fred Göbel, Speyer (DE)

(21) Appl. No.: 18/288,628

(22) PCT Filed: Apr. 28, 2022
(86) PCT No.: PCT/IB22/53956

§ 371 Date: Oct. 27, 2023

(30) Foreign Application Priority Data
Apr. 28, 2021 (DE) 10 2021 002 240.8

(51) Int. Cl. A61M 25/10

(52) U.S. Cl. CPC A61M 25/1002; A61M 25/1029; A61M 2025/1031; A61M 2025/1075; A61M 2205/0216

(57) ABSTRACT

The invention relates to a device for supplying and/or draining substances in a minimally irritating, tissue-compatible manner that is preferably adapted to the movements of the body and its organs, the device comprising a balloon, which can be placed in an interior space of the body and filled from outside the body, which balloon (optionally together with a supplying and/or draining tube or shaft segment that carries the balloon) surrounds a fillable compartment, to which a tube or shaft-like segment is attached which connects the interior space to the body surface; wherein the balloon is produced by blow moulding from a tube blank made of a multilayer film-like material; wherein the tube blank is extruded in multiple layers: wherein, in addition to at least one elastically deformable layer of polyurethane (PUR), at least one non-elastically deformable, odour- and/or media-tight barrier layer of ethylene vinyl alcohol copolymer (EVOH) or of polyvinylidene chloride (PVCD) or of polyamide (PA) or of a thermoplastic polyamide elastomer (TPE-A) is provided in the multi-layer material: and wherein the tube blank that has undergone blow moulding has been extruded in a previous separate method step, such that a ratio, required for the blow moulding process, of crystalline to amorphous material proportions is established in the layer of polyurethane. The invention also relates to a method for producing such a device.

### REFERENCE TO PENDING PRIOR PATENT APPLICATIONS

This patent application is a 371 national stage entry of pending prior International (PCT) Patent Application No. PCT/IB2022/053956, filed 28 Apr. 2022 by Advanced Medical Balloons GmbH and Fred Göbel for CATHETER-LIKE DEVICE FOR SUPPLYING AND/OR DRAINING SUBSTANCES TO OR FROM THE BODY OF A PATIENT, AND METHOD FOR PRODUCING SUCH A DEVICE, which patent application, in turn, claims benefit of German Patent Application No. DE 10 2021 002 240.8, filed 28 Apr. 2021.

The two (2) above-identified patent applications are hereby incorporated herein by reference.

### FIELD OF THE INVENTION

The invention relates to particularly simple constructions of supplying and/or draining catheter systems that remain in the body and to a method for producing these systems.

In particular, the invention relates to a device for supplying and/or draining substances in a minimally irritating, tissue-compatible manner that is preferably adapted to the movements of the body and its organs, the device comprising a balloon, which can be placed in an interior space of the body and filled from outside the body, which balloon—optionally together with a supplying and/or draining tube or shaft segment that carries the balloon—surrounds a fillable compartment, to which a tube or shaft-like segment is attached which connects the interior space to the body surface, wherein the balloon is produced by blow molding from a tube blank made of a multi-layer film-like material; the invention also relates to a method for producing such a device.

### BACKGROUND OF THE INVENTION

For balloon catheters designed for remaining in the body of a patient for longer periods of time, the volume of the respective filling medium received by the balloon must be held as constant as possible in the balloon throughout the course of the application. Depending on the physical and chemical characteristics of the balloon material, liquid or gaseous media may leak from the balloon component into the body or its respective environments during application. A discharge of the balloon caused by such a spontaneous loss of volume is usually accompanied by a partial or complete loss of the catheter's or the device's function.

In particular, balloon components made of silicone, even at higher wall thickness, are often capable only to a very limited extent to keep a liquid or gaseous fill volume, for example of water or air, constant within the balloon. Even for latex-based ballloons, a spontaneous volume discharge is not uncommon throughout the course of application.

For silicones as well as latex-like natural rubbers, the loss of volume can usually be explained by the typical porosity inherent to the material, whereby the permeability of the porous wall increases with the expansion of the balloon envelope.

Balloon materials having a distinct molecular polarity, such as polyurethane, do not have a porous wall structure, however, they still exhibit high permeability when filled with polar media of small molecular size, such as water in particular. The polar charged water enters into the likewise polar charged PUR material of the balloon envelope and, in the presence of correspondingly acting concentration gradients, exits on the other side of the balloon wall. Under certain circumstances, the “solution” or migration of water through a PUR-based balloon envelope may lead to an accumulation of water inside the balloon, which may compromise the functionality and security of tracheal ventilation catheters in a risk-relevant manner. It is known that water droplets that enter into the small-diameter supply line leading to the balloon (cuff) of a tracheal tube can temporarily interrupt the free communication between the tracheally sealing balloon and a pressure sensing or pressure regulating device external to the body.

In addition, the migration of water through PUR-based balloon envelopes can be problematic when housing electronic measuring or sensing components that are sensitive to water.

If a PUR-based balloon is placed in an aqueous solution or in aqueous body fluids having an osmotically acting gradient towards the aqueous filling medium of the balloon, concentration-dependent displacements of water across the balloon envelope out of the balloon and into the environment surrounding the balloon can occur. For example, when placing a water-filled PUR balloon into the bladder, such an osmotically driven migration of water out of the catheter balloon into the urine carrying salts and organic molecules may occur.

The spontaneous evacuation of a catheter balloon filled with gas, for example with air, into the body medium surrounding the balloon represents a particular challenge for thin-walled balloon materials. When a balloon made of PUR with a wall thickness in the micrometer range is placed into the bladder of a patient, usually a reduction of the fill volume occurs within a few days, which may, to a large extent, lead to a loss of the catheter balloon's retaining function.

Similar issues arise with tubes for supplying and/or draining media into and/or out of the human body, if these media are not to leak through the respective tube's wall, for example, in order to avoid unpleasant smells.

In particular, the permeability of the respective balloon envelope for aqueous and gaseous media correlates with its wall thickness, aside from its molecular polarity. The thinner the balloon envelope, the easier it is for the respective media to pass through. For many applications of catheter balloons, the specific catheter function depends particularly on maximally thin walls of the balloon envelope. The long-term placement of such membrane-like thin-walled balloons within the body thus usually requires close, intermittent control and a correction of the filling volume required for proper function. Therefore, the user expects balloon films with a special film structure and/or a combination of single layers with specific physical and chemical properties that prevent the spontaneous evacuation of the balloon via migration and osmosis effects.

### SUMMARY OF THE INVENTION

This leads to the object of the invention to implement a catheter-like supplying or draining device that remains within the body for a prolonged period of time and is preferably provided for the combined retention and sealing of the device and/or provided with a special transluminally sealing straightening mechanism, such that it may be manufactured in an economically advantageous way, may be applied over an extended period of time, and is able to adapt optimally to changes of the body lumen, if necessary.

The aforementioned object may be attained with a generic, catheter-like supplying or draining device, where a balloon that can be placed within an interior space of the body and filled from outside the body surrounds—optionally together with a supplying and/or draining tube or shaft segment that carries the balloon—a fillable compartment, with which a tube or shaft segment is joined, which connects the interior space with the body surface, where the balloon is manufactured by blow molding from a tube blank made of a multi-layer film-like material, the tube blank being coextruded in a multi-layered fashion and in addition to at least one elastically deformable layer of polyurethane (PUR), at least one not elastically deformable, odor- and/or media-tight barrier layer of ethylene-vinyl alcohol copolymer (EVOH) or of polyvinylidene chloride (PVCD) or of polyamide (PA) or of a thermoplastic polyamide elastomer (TPE-A) is provided in the multi-layer material, where the different layers are manufactured by means of coextrusion and the total thickness of all elastically deformable layers corresponds to at least 1.5 times the total thickness of all not elastically deformable, odor- and/or media-tight barrier layers.

Therefore, the present invention describes simple solutions for the manufacturing-optimized, cost-effective production of catheter-like supplying or draining devices that remain within the body for prolonged periods of time and are in particular equipped for the combined retention and sealing of the device.

In order to prevent spontaneous evacuations of particularly thin-walled balloon walls, the invention proposes a multi-layered construction of the film wall, where a specific material layer with special gas- and/or liquid-tight separation properties is integrated into the balloon envelope. Materials based on EVOH are particularly suitable for the manufacture of barriers with these properties. EVOH, also known as EVAL (ethylene-vinyl alcohol copolymer), has a very high gas sealing efficiency even at very low layer thicknesses of, for example, 4 to 10 micrometers, and also acts in an efficiently sealing manner against the permeation and/or migration of water or water vapor. In addition to the efficient barrier effect, EVOH is, as an essential further prerequisite for the application in the context of the present invention, sufficiently plastically deformable in order to be able to be formed or reshaped into a defined, balloon-like shape in combination with layers made of PUR, for example.

In order to form film bodies made of PUR with particularly thin walls, extruded tube material is needed that is usually manufactured in a separate manufacturing step prior to blow molding. The film material used within the scope of the invention is preferably made of 3 layers, where the outer and inner tube layers are made of PUR and the EVOH-based separation layer is received in the middle between the two PUR layers. Preferably, in this material combination, adhesion-promoting intermediate layers are omitted which connect the PUR and the EVOH in a mechanically durable way and without delamination of individual layers.

Another barrier-effective material is PVDC (polyvinylidene chloride). It has equally efficient sealing properties against oxygen and water vapor and is thus a preferable multifunctional separation layer within the scope of the invention. In addition, it may, for example, be extruded in multi-layered fashion together with PUR as a tube blank and plastically formed or “stretched” via blow molding. The wall thicknesses necessary for the barrier are in the one-digit micrometer range, just as for EVOH.

To achieve balloon envelopes with thicknesses in the micrometer range manufactured via blow forming from PUR or PUR-containing materials, the tube blank extruded in a separate process undergoes a certain axial stretching, is subsequently heated in a mold cavity and formed into a balloon-like body or expanded into this cavity, respectively, by applying blowing pressure. The axial and radial stretching of the tube material gives the formed balloon a certain polymeric orientation which provides high mechanical strength and form stability. In the moment of the combined axial and radial expansion of the tube blank to the balloon, an orientation structure of the amorphous polymer components arises that is correlated and parallel to the respective expansion, whereby said polymer components are aligned in proportional linear fashion. The alignment of the polymer chains is fixed by subsequent cooling in the formed state, where the formed mass of the balloon in the heated state is kept up to a retraction of about 5 to 10%.

In its simplest embodiment, the technology according to the invention describes the continuous forming of all components of the head unit of the device from a single tube blank. In addition to the forming of the head unit from a blank, the draining tube unit that adjoins the head unit extracorporeally may also be formed in the same production step with the head unit. The special feature according to the invention during the manufacture of the head portion is the specific implementation and arrangement of (corrugated) segments having a wavy profile in the area of the intracorporeal transluminal segment of the device. The specific profile shape of the intracorporeally retracting and/or transluminal supplying or draining tube ensures that both within the segment to be retracted, for example within the rectum, and within the transluminal segment, for example within the anal canal, a sufficiently strong elastic self-straightening effect of the supplying and/or draining lumen is generated, such that, for example, the contents of the gastrointestinal tract may drain to the outside with as little impediment as possible.

Because at least one of the supplying and/or draining tube structure, which carries the balloon or balloon portion retracting in the respective interior space or organ, and/or the transluminal segment of the device, which forms the access path to the interior space, has a corrugated, annular or helical-like profile shape, the device is equipped with a special transluminally sealing straightening mechanism that optimally follows and adapts to changes in the anal canal. In particular, the invention focuses on the aspect of economically advantageous manufacturability, where all components that are required for manufacturing the transanally positioned head unit of the device are preferably manufactured from a single tube blank in a single operation.

The respective elastic deformation and self-straightening properties of the intracorporeal, in particular rectal, and the transluminal, in particular transanal tube portions result, on one hand, from the elastic properties of the material used and its wall thickness, and, on the other hand, from the respective geometric design of the corrugation profile.

The straightening properties supported by the profile shape allow reducing the tube wall thickness in this segment to a film-like thin range in a particularly advantageous fashion for atraumatic application. This reduces the probability of structural and functional damage, for example to the anal sphincter. The combination of material and shape according to the invention allows adjusting and reproducing the manufacturability of the mechanical deformation and straightening properties within narrow limits.

Wall profiles are described that are typically manufactured via blow forming processes, where a previously extruded, relatively thick-walled tube blank is transformed into a film tube with relatively thin walls, or a tube blank with relatively small lumen is expanded to a relatively larger operating diameter by applying blowing pressure, respectively, and, in a heated state, is nestled into a correspondingly profiled mold wall. The invention encompasses comparable profile structures, such as those that may also be manufactured by means of a single- or multi-layered immersion process or an injection molding process.

The tube blanks used within the scope of the invention preferably consist of a multi-layer material, where material layers having elastic deformation properties, such as those preferably provided by thermoplastic polyurethanes (TPU), are proportionally predominant.

In particular, odor- and/or media-tight barrier layers may be integrated into the multi-layered structure, which is advantageous for the respective function of the catheter. For example, the tube film blank may have a sandwich-like structure, with PUR layers being the inner and outer sides and a middle layer made of EVOH or PVCD. In addition to the elastically acting PUR, also additional inelastic materials may be added, if these are needed to achieve specific surface properties of the product, for example. As an option to elastic materials, PVC- and PE-based material types may also be used, however less preferred.

In case of an integral design of the supplying and/or draining extracorporeal tube element adjoining the head unit, the corresponding tube element preferably has such thin walls that it folds in a radially inward direction or collapses into a flat, strip-like structure when an external force is applied and thus prevents compression-related lesions, in case the patient's body rests on the tube temporarily. In a preferred embodiment of the device, when the applied external force diminishes, the flatly collapsed tube segment spontaneously straightens in an elastic manner and reaches an at least partially open, partially rounded cross section. A corresponding straightening behavior may ideally be achieved by means of a single- or multi-layered composition made of polyurethane.

Due to its high mechanical strength and relatively low material compliance, PUR achieves a particular form stability of the structures formed to their respective full operating dimensions. In particular, this allows the option to use compressible gaseous filling media, for example the use of air. Further, these PUR-based balloon components allow the option of slackly filling the balloon without tension, because the anchoring and retaining stability of the balloon shape does not have to be maintained by continuous stretching of the balloon envelope, as is required, for example, with silicone-based balloon components. The PUR-based slackly and tensionlessly filled balloon body absorbs the force being applied and only assumes its shape specified during the manufacturing process when an external force is applied onto the balloon. Due to the preferably low elasticity of the balloon envelope, the respective functionally relevant, for example retaining, shape of the balloon is maintained when a corresponding physiological pressure or a tensile force away from the patient is applied.

For PUR support layers, thermoplastic, ester- and ether-based polyurethanes (TPU) are preferably used within the scope of the invention. A Shore hardness in the range of 80 A to 95 A and in the range of 55 D to 60 D is preferable. For example, TPU types available from Lubrizol (Pellethane 2363 series) or BASF (Elastollan 1100 series) may be used.

If support layers based on PVC are combined with a barrier layer on the basis of EVOH or PVDC, the previously described straightening properties typical for PUR are only marginally sufficient or cannot be achieved. However, with a proportional use of PVC, for example as the inner or outer support layer within the multi-layer tube envelope, the capability for elastic self-straightening of the rectal and transanal tube portions may be achieved by integrating an additional PUR layer into the film structure. Preferably, in this case, the employed PUR has a higher Shore hardness, for example in the range of Shore 95 A or also Shore 55 D to 65 D, and the wall thickness may be kept relatively thin relative to the PVC layer in order to maintain any desired advantages of PVC.

Within the scope of the invention, a PVC layer that faces the drainage lumen may be conceptually advantageous, since the barrier effect against water that is typical for PVC exceeds the achievable barrier effect of a PUR layer having the same wall thickness considerably. If, in particular, EVOH is used as the barrier layer, a maximally efficient protection from water molecules is advantageous, because its barrier efficiency is reduced when it is exposed to water.

PUR-based material layers provide particular mechanical stability to the multi-layered tube components described within the scope of the invention. Even thin-walled proportional PUR layers in the range of 10 to 30 μm impart sufficient tensile strength and tear strength, as well as cut resistance and puncture resistance, to the formed tube and balloon segments. In addition, corresponding PUR layers stabilize the blank when forming the blank into the forming tool.

Aside from EVOH or PVDC, also layers of polyamide (PA) or Pebax, a substance related to polyamide, may be integrated into a multi-layer film as an efficient odor barrier. In a preferred embodiment, the invention proposes thin-walled polyamide or TPE-A layers (e.g. Pebax®) in the range of approximately 10 to 20 μm that are combined with a PUR support layer. The achievable barrier efficiency is inferior to that of EVOH or PVDC.

The film combinations described within the scope of the invention may, for example, be manufactured by means of multi-layered film extrusion. Here, the tube blank used in the molding process may be extruded in primary fashion or it may also be processed from a flat multi-layer film into a tube blank moldable by blow molding.

### BRIEF DESCRIPTION OF THE DRAWINGS

Further characteristics, features, advantages, and effects of the invention will be apparent from the following description of the preferred embodiments of the invention and by reference to the Figures. In these:

FIG. 1a illustrates an exemplary balloon film tube formed by blow molding from a tube blank for manufacturing a catheter head unit according to the invention;

FIG. 1b illustrates the balloon film tube of FIG. 1a in a folded-back state closed towards the fillable compartment;

FIG. 1c illustrates a head unit based on the blank shown in FIG. 1a, where, in addition to the segments of the head unit, the extracorporeal supplying and/or draining tube segment is molded from a continuous tube blank;

FIG. 2a illustrates an embodiment of a tube blank where both the entire supplying and/or draining intracorporeal segment is provided with a corrugated or helical profile and the molded, retracting balloon segment optionally extends through the access path to the respective interior space or even beyond it;

FIG. 2b illustrates the balloon film tube of FIG. 2a in a folded-back state closed towards the fillable compartment;

FIG. 3 illustrates a molded film tube, where the intracorporeal tube segment comprises portions with differently shaped profiles;

FIG. 4a illustrates a molded film tube comprising, in the balloon supporting segment, an additional annular or tubular component stabilizing the lumen of the device;

FIG. 4b shows the balloon film tube of FIG. 4a in a folded-back state closed towards the fillable compartment;

FIG. 5 illustrates an exemplary corrugation profile according to the invention based on a shaft tube having an inner diameter of 12 to 14 mm;

FIG. 6 illustrates an exemplary multi-layered structure of a tubular film blank;

FIG. 7 illustrates another exemplary multi-layered structure of a tubular film blank;

FIG. 8 illustrates an exemplary schematic of a wall of a tube blank used for blow molding, where the blank comprises a particular combination of three concentrically extruded material layers with a centered separation layer;

FIG. 9 illustrates a three-layered balloon body formed by means of blow molding from the tube blank described in FIG. 8;

FIG. 10 illustrates a partially sectional cross section through a balloon body according to another embodiment of the invention having a special additional inner layer made of TPE-based material;

FIG. 11 illustrates a balloon body having a special four-layered structure in a view corresponding to FIG. 10;

FIG. 12 illustrates a balloon body comprising a special combination of a thermoplastic polyurethane and two additional barrier layers in a view corresponding to FIG. 10;

FIG. 13 shows a schematic, partially sectional cross section of a structure of a balloon envelope according to the invention from two combined material layers;

FIG. 14 shows a transversal section of a secretion-conveying, channel-like formation, as it develops in the case of residually dimensioned, sealing and/or tamponading balloon bodies in a lumen smaller relative to the balloon;

FIG. 15 illustrates a corresponding secretion-conveying, channel-like formation, which increases in the shape of a drop when the filling pressure is reduced and opens in a U-shape when the filling pressure is further reduced, in a view corresponding to FIG. 14;

FIG. 16 shows a schematic, perspective view of a tracheal tube cuff, wherein the channel-like formations bridge the balloon body from one end-face side to the other;

FIG. 17 illustrates a 2-layer embodiment of a balloon wall, the supporting PUR layer being combined with a water vapor-tight barrier layer made of PVDC, in a view corresponding to FIG. 13;

FIG. 18 illustrates a 3-layer embodiment of a balloon wall, the supporting PUR layer being combined with a middle barrier layer made of PVDC and/or EVOH, and a PVC layer that attenuates the elastic straightening properties of PUR, in a view corresponding to FIG. 13;

FIG. 19 illustrates a qualitative comparison of two types of balloons formed from elastic PUR, one of the design types being combined with a layer of PVC that modifies the elastic properties of PUR;

FIG. 20 depicts a side view of the distal end of an embodiment of a urinary catheter according to the invention prior to its insertion into the bladder;

FIG. 21 depicts a sectional view through a bladder into which the catheter of FIG. 20 is intravesically placed in situ;

FIG. 22 illustrates a tracheal tube according to the invention having a continuous supply line integrated into the shaft, in combination with an external device that regulates volume and pressure, respectively, and a flow-directing device;

FIG. 22a depicts a section through FIG. 22 along the line IIa-IIa;

FIG. 23 illustrates a modified embodiment of a tracheal tube according to the invention having a one-chambered balloon that seals tracheally and tamponades subglottally;

FIG. 23a illustrates yet another modified embodiment of a tracheal tube according to the invention comprising a two-chambered arrangement and a tracheally sealing and a sub- to supraglottally tamponading balloon;

FIG. 24 illustrates a supply line to the balloon of a tracheal tube according to the invention comprising a combined valve and throttle mechanism;

FIG. 25 illustrates an exemplary control and reservoir component, respectively, for a tracheal tube according to the invention;

FIG. 25a illustrates a pressure-volume curve associated with the component described in FIG. 25;

FIG. 25b illustrates a reservoir and control balloon configured for two-chambered systems;

FIG. 26 illustrates a tracheal tube according to the invention with optimized supply line comprising a sensor element in the region of the tracheally sealing balloon segment and an electro-mechanical control unit arranged in a control loop with the sensor;

FIG. 27 illustrates another tracheal tube according to a modified embodiment of the invention comprising a sensor element in the region of the tracheally sealing balloon segment and an electronic control unit arranged in a control loop with the sensor; and

FIG. 28 illustrates a trans-esophageal probe with a connected control unit.

### DETAILED DESCRIPTION OF THE PREFERRED EMBODIMENTS

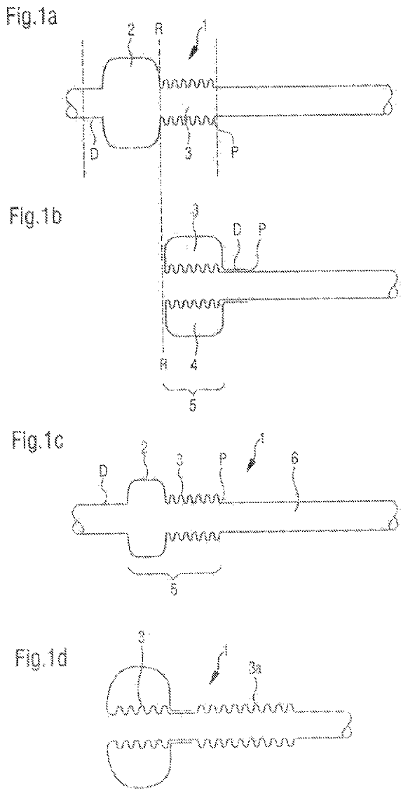

FIG. 1a illustrates a schematic longitudinal section of an exemplary balloon film tube 1 prepared by thermal forming from a continuous tube blank and comprising a spherically expanded balloon portion 2, as well as a shaft tube portion 3 having an wavy, annularly or helically corrugated profile and closing the balloon towards the closed compartment. The molded tube film is folded backwards in the plane R. The distal balloon end D is connected to the proximal end P in a flatly, tightly sealing manner. The corrugated shaft tube portion 3 provides radial stability to the tube film within the closed compartment that withstands filling pressures within the balloon up to 200 mbar, preferably in the range of 50 to 100 mbar, and counteracts a radially inward deformation, invagination, and a collapse of the tube film, respectively.

The compartment is filled from outside the body via a separate tubular supply line, that may, for example, be installed into the joining region between the balloon ends D and P.

FIG. 1b shows a balloon tube film 1 corresponding to FIG. 1a in a state closed towards the fillable compartment 4.

FIG. 1c illustrates a balloon film tube as shown in FIG. 1a, where the extracorporeal portion of the catheter device is molded from the same tube blank as the intracorporeal portion of the device.

FIG. 1d discloses a balloon film tube 1 corresponding to FIG. 1a in a state closed towards the fillable compartment 4, where the corrugated profile design of the tube film is continued across the balloon supporting portion 3 positioned within the cavity as a transluminal and/or transanal portion 3a.

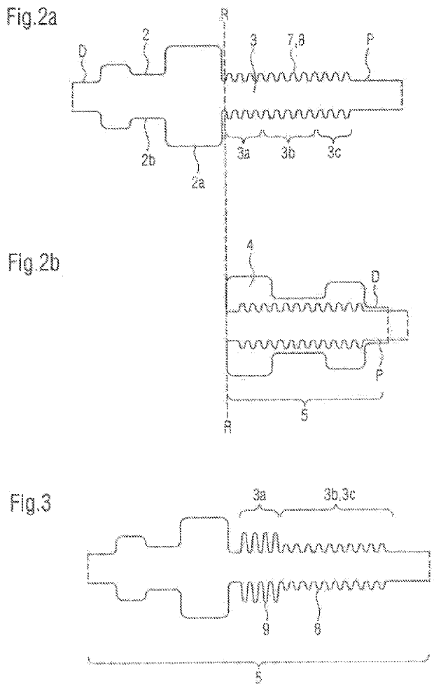

FIG. 2a shows a molded tube film, analogous to FIG. 1a, where the balloon portion 2 has a specific barbell-like or optionally mushroom-like geometry and the balloon portion 2 extends into or through the anal canal or even protrudes from the anus, and where the balloon portion is folded back over a corrugated shaft tube 3, the distal balloon end D being connected, for example by flat gluing or welding, with the proximal shaft tube end P to a tightly sealing space that is fillable from the outside. Both the rectal portion 3a and the transanal portion 3b and the optional, preanally placed portion 3c of the shaft tube are provided with an annular or helical-like wall profile 7 according to the invention. The wall of the shaft tube consists of thermoplastic PUR on the basis of ether with a Shore hardness of 85 A to 90 A, having a wall thickness in the range of 500 μm to 600 μm and an inner shaft diameter in the range of 15 to 17 mm. The outer shaft diameter is correspondingly in the range of 16 to 18.2 mm. The shaft tube comprises outwardly directed, annular or helical, U-shaped extensions 8 extending from the outer shaft diameter with an amplitude in the range of 2.0 to 3.0 mm and an apex distance in the range of 3.0 to 5.0 mm. The width of the U-shaped extensions at the base is 1.5 to 3.0 mm. In this embodiment, the corrugated profile 7 extends over a total length of 70 to 100 mm.

When the shaft tube segment and the balloon segment are molded from a single blank extruded in a preceding step, the balloon wall thickness in the region of the largest rectal balloon diameter 2a in the range of 55 to 75 mm is approximately 40 to 80 μm, in order to achieve the shaft and corrugation dimensions that are necessary for the required straightening properties according to the invention. In the central region of the balloon 2b with a diameter of approximately 25 mm, the balloon wall thickness is approximately 200 to 300 μm.

FIG. 2b illustrates the tube film shown in FIG. 2a in the state closed towards the fillable compartment 4. In the illustrated embodiment, the entire intracorporeal portion of the stool draining lumen of the device is housed inside the balloon.

FIG. 3 illustrates another embodiment of a head unit 5 derived from FIG. 2a that comprises differently designed portions with a corrugated profile in the region of the shaft tube segment enclosed by the dumbbell-shaped balloon. The figure shows U-shaped formations in the region of the rectally placed extension 3a that are denser and higher compared to the corrugation in the transanal segment 3b. While the shaft and corrugation dimensions in the transanal segment 3b correspond to those of FIG. 2a, the shaft tube comprises convex U-shaped extensions 9 with a height of, for example, 3.5 to 5.0 mm and a width of 2.0 to 2.5 mm in the distal segment 3a. The space between the U-shaped extensions 9 at the base is 1.0 to 2.0 mm. The higher and denser corrugation profile provides the shaft tube in this segment with a higher, lumen-preserving stability, which counteracts a radial collapse of the segment in a rectal position and sufficiently counteracts the force onto the balloon arising within the respective cavity, respectively, and which keeps the stool-carrying and draining mouth of the device facing the rectum open, respectively.

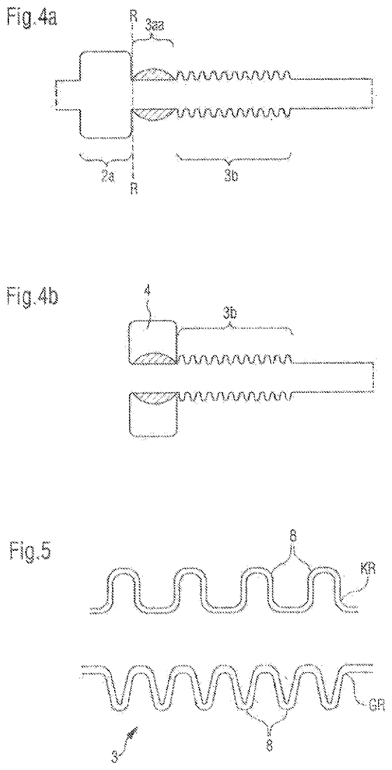

FIG. 4 is another embodiment of a device according to the invention, where the transluminal or transanal portion 3b of the shaft tube is strengthened by a wavy corrugation of the tube wall in a way that straightens and/or maintains the lumen, but where the tube segment 3a placed within the cavity that closes the balloon towards the fillable compartment does not have a profile shape that straightens and/or maintains the lumen. In this specific embodiment, the portion of the supplying and/or draining catheter-like device without corrugation is held open by means of a sleeve-like cylindrical element 3aa stabilizing the central lumen, the element being manufactured separately. In the preferred embodiment, the element is installed into the interior space 4 of the folded-back fillable compartment positioned in the rectum, where the inner surface of the cylinder is flatly connected to the outer surface of the balloon film, for example by adhesion with a solvent. Alternatively, the lumen-maintaining element may also be distally inserted into the mouth of the central channel of the folded-back film tube and fixed there.

FIG. 5 depicts another exemplary embodiment of a corrugated shaft tube 3, starting from an inner diameter D of the shaft tube of 12 to 14 mm. The wall of the shaft tube consists of a thermoplastic PUR on the basis of ether with a Shore hardness of 85 A to 90 A and a wall thickness in the range of 400 μm to 500 μm. The outer shaft diameter is correspondingly approximately 13 to 15 mm. Starting from an outer shaft diameter the shaft tube comprises convexly directed, U-shaped extensions 8 with an amplitude height of 1.0 to 2.0 mm and a width of 1.5 to 2.5 mm that transition, in the shaft region, with a small radius KR of approximately 0.25 mm into the shaft tube. The axial spacing of the U-shaped extensions to the shaft tube is 1.5 to 2.5 mm at the base. Alternatively, extensions 8 that are directed outwards in a convex fashion may also transition into the shaft tube with a large radius GR of approximately 1.0 mm or have an approximately sinusoidal shape.

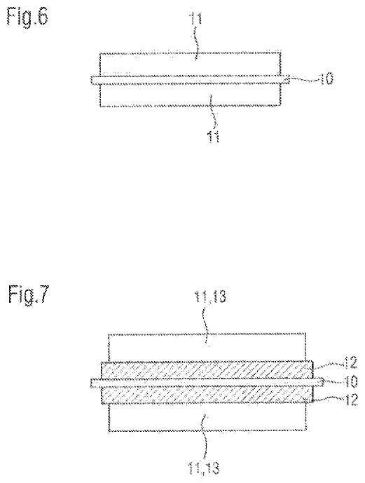

FIG. 6 depicts an exemplary layer construction of a molded tube film blank 2, comprising a middle barrier layer 10, preferably made of a material based on EVOH, for example of the type Eval, available from the manufacturer Kuraray, Japan, with a layer thickness of 5 to 50 μm, as well as adjoining layers 11 of a support material on both sides, preferably made of PUR, for example of the type Elastollan 1185 A, available from the manufacturer BASF, with a respective layer thickness of, for example, 20 to 100 μm.

FIG. 7 illustrates an alternative film tube construction where the inner barrier layer 10 with a thickness of 5 to 50 μm, preferably made of EVOH, is sandwiched between a polyamide layer (PA) 12 on both sides with a wall thickness of 10 to 50 μm. EVOH and PA enable well adhering coextrusion behavior. Again, PUR layers 11 or PVC layers 13 adjoin the PA layers 12 on one or both sides, which increases the total wall thickness to approximately 50 to 500 μm. The advantage of this combination is on one hand the increased stability with the help of the PA portion, on the other hand the compatible coextrusion properties of the materials used which makes the use of any additional adhesive layers (tie layer) unnecessary. In order to provide elastic straightening properties to the film tube, one or more PUR layers may be added, preferably on the outside.

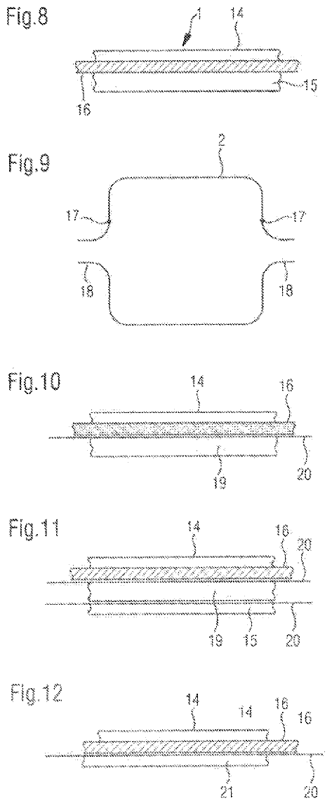

Similar to FIG. 6, FIG. 8 shows the wall structure of a multi-layer extruded tube blank 1, whose wall is composed of three material layers, where the outer layer 14 and the inner layer 15 are made of a thermoplastic polyurethane (TPU) or a material containing or based on polyurethane. In particular, TPU of an ester or ether-based type, preferably with a Shore hardness of 85 A to 95 A, is recommended. The total layer thickness of the two TPU layers 14 and 15 in the tube blank may herein be in a range of 80 μm to 200 μm, preferably in a range of 100 μm to 180 μm.

In contrast, the intermediate layer 16 is made of a barrier material, preferably of the type EVOH, that has particularly efficient barrier properties in terms of avoiding the passage or transfer of gaseous air components and water molecules from one side to the other side of the multi-layer film combination. In a combined multi-layer extrusion with polyurethane, specific types of EVOH, such as those available by the manufacturer Kuraray Co., Ltd., enable the omission of adhesion-promoting material layers, so-called “tie layers,” which are otherwise typically used to strengthen the connection of the individual layers when combining different types of materials. The total layer thickness of the barrier layer 16 of the tube blank may herein be in a range of 10 μm to 60 μm, preferably in a range of 20 μm to 40 μm.

The extrusion of the tube blank is thus reduced from five to only three layers, which is very advantageous for the manufacturing process. The extrusion of smaller tube blank diameter of, for example 3 to 10 mm, preferably of 3 to 7 mm, thus becomes critical due to the omission of the two tie layers.

The thicknesses of the various tube layers is reduced compared to the tube blank by means of the subsequent blow molding step during the radial stretching of the tube. Thereby, the wall thickness of the barrier layer 16 is reduced, for example, to a range of 3 μm to 10 μm, preferably to a range of 4 μm to 8 μm. At the same time, the reduction of the support layers 14, 15 to a total thickness of these layers 14, 15 of, for example, 7 μm to 20 μm, preferably to a total thickness of these layers 14, 15 of, for example, 12 μm to 15 μm. This results in a total thickness of the final balloon structure between 10 μm and 30 μm. In order to maintain the flexible film properties in the respective molded balloon- or tube-like structure, the dimensions of the total wall thickness proportions of the barrier layer 16 is desired such that the total wall thickness proportion of the one or all barrier layers 16 with respect to the total wall thickness of the molded balloon- or tube-like structure 2 is between one third and one eighth, preferably between one quarter and one seventh, more preferably between one fifth and one sixth.

Correspondingly, PVDC may also be positioned in the middle between two polyurethane layers. However, an “immediate,” direct adhesion of PVDC to PUR is not possible, which necessitates an additional layer with an adhesion promoter. In contrast to EVOH, which is soluble in an aqueous environment, PVDC may be exposed directly in an aqueous environment, which turn can reduce the number of material layers once more to a total of three technically feasible layers.

In order to ensure an optionally permanent joining or adhesion with a solvent when the molded balloon body is installed onto a shaft element supporting the balloon, the inner polyurethane layer 15 of the balloon tube film 1 facing the shaft element supporting the balloon preferably has a thicker wall thickness than the outer PUR layer 14.

FIG. 9 describes a balloon body 2 made of the raw material of FIG. 8. Due to the middle barrier layer 16 made of EVOH or PVDC and its flat, fixed connection to the adjoining support layers 14, 15 made of PUR, the EVOH or PVDC, respectively, which deforms in a ductile manner in contrast to PUR, correspondingly deforms when the elastic support material polyurethane adjoining the EVOH or PVDC on both sides is deformed. The wall thickness of the EVOH or PVDC portion thus decreases nonlinearly from the axial center of the blank or the balloon to its largest diameter when the balloon corpus is radially molded, as would be expected when radially stretching a purely ductile material, rather the thinning that effectively results corresponds to that of the elastically deforming support or composite material. Therefore this allows, in both regions between the inflection points 17 of the balloon shoulder radii, where the longitudinal curvature of the balloon 2 transitions from concave to convex, an approximately uniform thickness of the EVOH or PVDC-based barrier layers such as that, which also results in the PUR layers sandwiching the middle separation layer. A pointwise, line-shaped or areal weakening of the barrier function of the balloon envelope may thus be avoided to a large extent.

In order to ensure this, the ratio of the minimal diameter in the region of the shaft ends 18 beyond the two inflection points 17 to the maximal diameter of the balloon 2 between the two inflection points 17 should not exceed 1:8 if possible, a ratio of maximal 1:5 is preferred.

In the exemplary case of a tracheal tube, the total diameter of the tube blank could preferably be between 4 to 10 mm, for example approximately 7 mm, with a preferable total wall thickness of the tube blank between 80 μm and 180 μm. The barrier layer 16 would account for 20 μm to 30 μm of the total wall thickness. While these thicknesses in the non-deformed balloon ends are maintained also in the molded balloon structure, in the region of the balloon 2 radial stretching in a ratio of approximately 1:5 or less occurs combined with an axial stretching of less than 1:1.5, which is necessary during the molding process for technical reasons, and there the total wall thickness of the final product is in a range of 10 μm to 25 μm, of which 3 μm to 5 μm represent the wall thickness of the barrier layer 16.

In the exemplary case of a urinary catheter, the total diameter of the tube blank could preferably be between 3 to 5 mm, for example approximately 4 mm, with a preferable total wall thickness of the tube blank between 70 μm and 140 μm. The barrier layer would account for 20 μm to 35 μm of the total wall thickness. While these thicknesses in the non-deformed balloon ends are maintained also in the molded balloon structure, in the region of the balloon 2 stretching in a ratio of approximately 1:7 or less occurs, and there the total wall thickness of the final product is in a range of 10 μm to 20 μm, of which 3 μm to 5 μm represent the wall thickness of the barrier layer 16.

FIG. 10 illustrates a specific concept of a wall structure, where the inner layer of the tube blank 1 or the balloon body 2 molded from it consists of a material 19 on the basis of TPE.

In this embodiment, the wall is also comprised of the following combination of layers (from outside to inside): PUR-EVOH-promoter-TPE, with a promoter layer 20. The specific combination ensures that the TPE inner layer 19 provides a technically simple connection with a support component on TPE basis receiving the balloon 2. Further, a balloon 2 manufactured in this way may be provided with barrier properties against water vapor that are typical for TPE.

Similar to FIG. 10, the invention utilizes the specific elastic, uniform deformation properties of polyurethane, where the wall thicknesses of the segment between the inflection points 17 of the shoulder radii are relatively constant during the radial expansion of the balloon envelope 2. The PUR layer 14 thus serves as support for the molding of the non-elastic EVOH layer 16 and the elastic TPE layer 19. In contrast to blow molding of PUR, balloon bodies 2 with a uniform, particularly thin wall thickness cannot be achieved when blow molding TPE. The option for dimensionally stable thinning of the PUR portion to very low layer thicknesses allows to increase the relative proportion of the TPE layer 19 in the overall wall and to bring out the specific vapor barrier properties of the TPE. FIG. 11 illustrates a 4-layer structure complementary to the structure of FIG. 10, where an additional layer 15 made of PUR adjoins the inner TPE layer 19, possibly after another promoter layer 20. This integrates the option of adhesion via solvent onto a shaft component supporting the balloon 2, and the two middle layers, the barrier layer 16 and the TPE layer 19, are sandwiched on both sides by the supportingly molded support material 14, 15. The wall structure is thus (from outside to inside) as follows: PUR-EVOH-promoter-TPE-promoter-PUR or PUR-promoter-EVOH-promoter-TPE-promoter-PUR. The layer thicknesses in the molded balloon 2 are for example as follows: PUR (5-7 μm)—EVOH (1-5 μm)—promoter (1-3 μm)—TPE (5-15 μm)—promoter (1-3 μm)—promoter (1-3 μm).

FIG. 12 shows a balloon body comprising an outer layer 14 made of a thermoplastic polyurethane, a directly adjoining barrier layer 16 made of EVOH without adhesion promoter and a thereto adjoining layer made of promoter 20 and PVDC 21. The layer thicknesses in the molded balloon are for example as follows: PUR (5-7 μm)—EVOH (1-5 μm)—promoter (1-3 μm)—PVDC (1-3 μm).

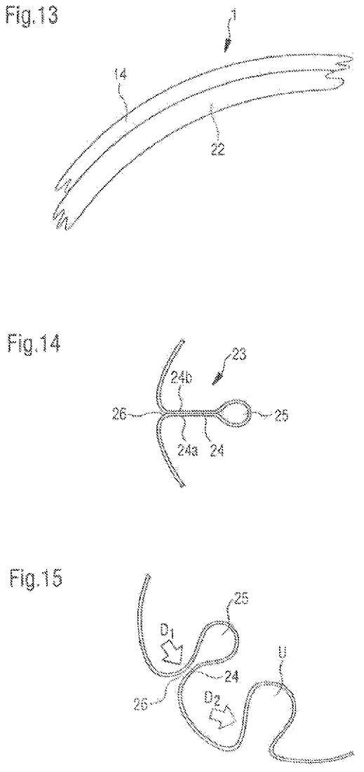

FIG. 13 illustrates a schematic embodiment of an exemplary 2-layered construction of a balloon tube film 1 according to the invention, where preferably the outer material layer 14 facing the respective lumen or cavity is made of thermoplastic PUR of the type Elastollan 1100 having a Shore hardness of 90 A and a proportional wall thickness of 5 to micrometers. Preferably, the material layer 22 facing the interior space of the balloon 2 is made of a PVC having a Shore hardness of 70 A and a proportional wall thickness of to 20 micrometers. The two polymers are preferably manufactured directly, that is, without an adhesion-promoting intermediate layer, by means of a coextrusion process in a flatly connected fashion that firmly adheres them together. On one hand, the 15 to 20 micrometer thick PVC layer 22 counteracts the elastic straightening of the PUR layer portion 14 folded into an eyelet-like formation in an attenuating manner that reduces the speed and the amount of the straightening process. On the other hand, the PVC layer portion 22 reduces the passage or migration of polar substances through the described PUR/PVC layer combination and thus reduces undesired condensation and accumulation effects of fluids within the interior space of the balloon 2, in particular of water.

Within the scope of the invention, the wall layers 14, 22 made of PUR and PVC may also be arranged in the layer combination in such a way that the PVC layer is disposed on the outside of the balloon and accompanied by an inner PUR layer 15.

Aside from 2-layer balloon walls, 3-layer embodiments are possible, where a PUR layer 14, for example, is sandwiched by two PVC layers 22. With a total wall thickness of 30 micrometers, the distribution of the individual layers may be, for example 12 μm PVC on the outside, 6 μm PUR in the middle and 12 μm PVC outside. This embodiment is particularly advantageous to limit undesired migration effects of polar substances, such as water, for example.

FIG. 14 depicts a schematic transversal section of a secretion-conveying invagination 23, where wrinkle-like structures develop due to infolding of the excess balloon wall 1 within a residually, that is, excessively molded sealing and/or tamponading balloon body 2 during its placement within a lumen or space that is smaller in relation to the residually dimensioned balloon 2. Particularly for cyclically changing filling pressures within the balloon 2, a typical, spoke-like arrangement of such infoldings 23 extending from the circumference towards the center of the balloon 2 arises during the course of application.

The invaginations 23 each have a web-like, flatly closed portion 24, while an eyelet-like formation 25 forms at the blind end of each invagination facing the balloon center. In the region of the forming eyelet, the wall 1 of the balloon envelope 2 folds over by 180 degrees, whereby a pronounced, opening effect on the eyelet-like formation is generated by the elastic straightening properties of the PUR layer integrated into the wall. The particular effective sealing effect of the balloon at a certain point in time results as a function of the size of the cross-sectional area of the particular eyelet-like formation, and of the greatest possible avoidance or reduction of cyclic jumps in eyelet diameter. Due to capillary effects on secretions situated within the eyelet, small cross-sectional areas of the eyelet act in a flow-inhibiting manner, up to a complete stasis of the secretion or of the eyelet contents. The effect of inhibiting the free flow of secretions is lost with increasing dilation or enlargement of the cross-sectional area of the eyelet.

In addition to the particular property for elastic straightening of the balloon wall that is folded over in an eyelet-like manner, the sealing-relevant cross-sectional area of the eyelet-like formation 25 is determined by the currently prevailing filling pressure in the balloon, which in particular acts onto the two wall layers 24a and 24b of the web-like portions 24 of the invagination 23 and flatly presses them together in a tightly sealing manner, an open lumen remaining in the area of the fold of the two wall layers, i.e., at the blind end of the invagination in question.

Preferably, the total wall thickness of a balloon 2 according to the invention does not exceed 30 μm. In a preferred embodiment of the balloon 2, the ratio of the wall thickness proportion of the PUR layer to the wall thickness proportion of the PVC layer is between 1:2 and 1:4, and is preferably 1:3.

If, for example, the filling pressure in the balloon 2 of a tracheal tube with the specific combination of layers 14, 22 described in FIG. 13 varies cyclically between 30 and 5 mbar due to the patient's own respiration, the cross section of the eyelets 25, which determines the sealing efficiency of the balloon 2, increases by 10% to 25%, but usually not more than 20%. The largest eyelet diameters of balloons 2 manufactured according to the invention that are measured within a respective eyelet-like formation are approximately 30 to 120 μm at a continuous filling pressure of 30 mbar, preferably approximately 40 to 80 μm.

When the filling pressure in the balloon 2 varies cyclically, for example with 20 oscillations per minute and pressure amplitudes or pressure extremes between 30 and 5 mbar, the sealing properties of the balloon 2 according to the invention are largely maintained for the specific application of tracheal sealing of secretions. Pump-like cyclically milking actions following the patient's own respiration onto the eyelet-like formation 25 or onto the channels extending from the eyelets 25, such as those that are described in the literature for thick-walled single-layer cuffs on the basis of PVC with a wall thickness of 70 to 120 micrometers, are largely absent for the tracheal tube cuff embodiment according to the invention.

FIG. 15 depicts an eyelet-like formation 25 corresponding to FIG. 14 for a filling pressure that is reduced relative to FIG. 14. If the filling pressure becomes less than a specific filling pressure D1 critical for the sealing effect, the entry region 26 on the base of the invagination 23 starts to open, and the eyelet-like formation 25 widens and elongates, beginning at the blind, inner end of the invagination towards the outer base 26 of the invagination 23. The web-like, tightly sealing segment 24 of the invagination 23 shortens correspondingly. If the filling pressure is further reduced to a value D2, the web-like segment 24 opens completely, and the invagination 23 transitions to a flat open U-shape U.

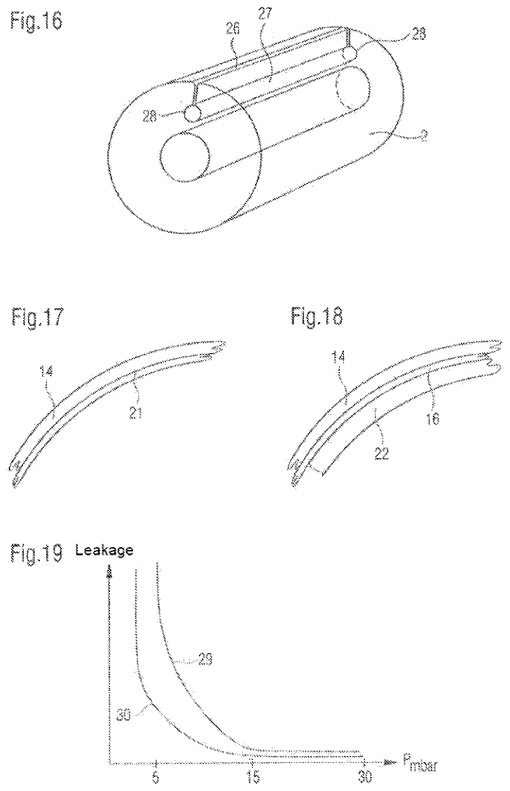

FIG. 16 shows schematically an exemplary cylindrical sealing balloon 2, for example applied as a secretion-sealing tracheal tube cuff, and shows channel-like formations 27 that extend from the eyelet-like turned-over formations 25 at the blind end of the respective invaginations 23. There, the channel-like formations 27 continuously extend from an end-face side 28 of the balloon cylinder 2 to the opposing end-face side 28 and, in many cases, assume an approximately parallel orientation to the cylinder axis of the balloon when cyclically varying filling pressures are continuously applied, and thus allow leakage of fluids or secretions from one end-face side to the other of the balloon, which sealingly encloses or space-fillingly tamponades a lumen or an interior space of a patient.

FIG. 17 illustrates a specific, 2-layered embodiment of a balloon wall 1, where the PUR layer 14 that stabilizes the balloon 2 is combined with a water vapor- and gas-tight barrier layer 21 made of PVDC. The PVDC layer 21 may be facing both the outside of the balloon 2 and its interior. Even in very thin layer thicknesses, PVDC has a very efficient water and gas sealing effect. The proposed combination thus provides the basis for the manufacture of particularly advantageous balloon walls with low total thickness in the range of 10 to 15 micrometers, which are advantageous in the sense of the smallest possible or most uniform, non-alternating cross section of the eyelet-like formation 25. Here, the PUR layer 14 has a layer thickness of 5 micrometers, for example, the PVDC layer 21, however, has a thickness of 5 to 10 micrometers, for instance.

FIG. 18 shows a specific 3-layered embodiment of a balloon wall, where the elastic PUR layer 14 is combined with a gas- and water vapor-tight middle barrier layer 16, for example made of PVDC or alternatively of EVOH, and a layer 22 for attenuating the straightening properties of PUR according to the invention, preferably made of PVC with low Shore hardness. With a total wall thickness of 25 micrometers, for example, the PUR layer 14 has a layer thickness of 5 micrometers, for example, the gas- and water vapor-tight barrier layer 16, made of PVDC for example, has a thickness of 5 micrometers, and the attenuating layer 22, for example made of PVC, has a proportional layer thickness of 15 micrometers.

FIG. 19 qualitatively illustrates with two graphs 29, 30, how the filling pressures in the interior space of the balloon 2 affect the sealing efficiency of the cross section of the eyelet-like formation 25 that is relevant for the respective sealing or tamponading catheter or device application. In a comparative approximation, a residually dimensioned, single-layer balloon 2 made of PUR having a wall thickness of 15 micrometers and forming radial invaginations of the residual balloon envelope, made of a material of the type “Elastollan 1190 A” in graph 29 is contrasted with a residually molded and dimensioned balloon 2 made of a two-layered material consisting of a combination of a PUR layer 14 and a PVC layer 22 according to the invention with a total wall thickness of 20 micrometers in graph 30, such as is described in FIG. 13 as an example for the technology. With approximately 20 cyclic fluctuations per minute between a pressure range of 30 mbar to 15 mbar, both balloon types exhibit a comparably efficient sealing effect, which corresponds to an almost complete seal. However, if the lower values of the pressure fluctuations reach into the range of 15 to 5 mbar, the two graphs 29, 30 diverge, where the cross section of the eyelet 25 of variant 29 is approximately 10 to 25% larger than that of variant 30 of the balloon 2 of FIG. 16. For the single-layer balloon of graph 29, the seal is completely lost in the pressure range below 5 mbar, which does not happen for the multi-layer balloon 2 according to the invention with a combination of a PUR layer 14 with a PVC layer 22 according to the invention until approximately below 3 mbar.

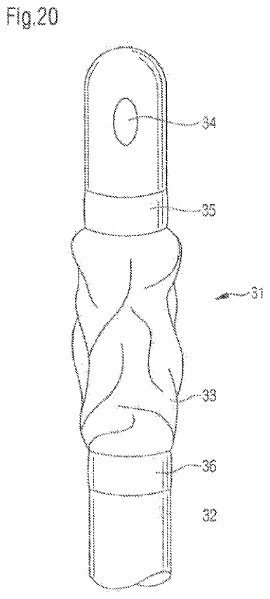

FIG. 20 depicts a side view of the distal end of an indwelling urinary catheter 31. The catheter 31 comprises a shaft 32 and a balloon 33 mounted to the shaft 32. Proximal to the distal end of the shaft 32 is an opening 34 that serves to drain urine or other liquids through the interior of the catheter 31. The balloon 33 is shown in its base state, that is, at rest and completely collapsed. Strips 35 and 36 are formed by the ends of the balloon 33 that serve as fluid-tight connections between the shaft 32 and the balloon 33. The strips 35, 36 may be connected with the shaft 32 itself by an adhesive, by means of ultrasound welding or by any other connection technique.

The empty or folded balloon 33 tightly abuts the wall of the shaft 32, whereby the film-like structure of the balloon 33 folds in random or preconfigured patterns. The balloon 33 is provided with two tube ends 35, 36 or shaft attachment pieces, respectively, which are fixed to the catheter shaft 32.

The shaft 32 of the catheter 31 may consist of an elastic material and may preferably be made of PUR or PVC. LDPE, LLDPE, SEBS, silicone or natural rubber are possible as well. Preferably, the catheter shaft 32 has a structure with three or two lumens.

With an advantageous choice of material, a shaft wall thickness of approximately 0.4 to approximately 0.8 mm, preferably approximately 0.4 to approximately 0.6 mm, is sufficient. The catheter shaft 32 is to maintain its stiffness and buckling safety, respectively, such as is necessary for insertion into the urethra in patient applications.

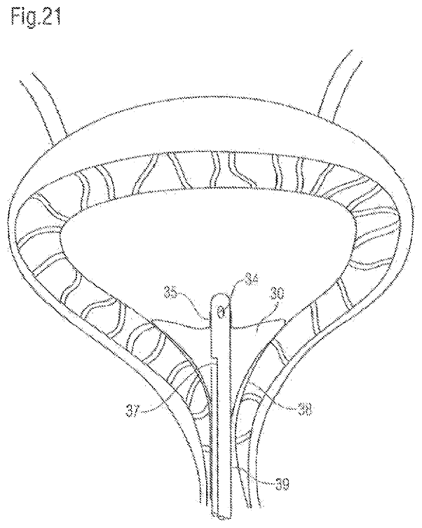

As shown in FIG. 21, the balloon 33 is shaped to its operating dimensions or residually and thus, in its operating state, only needs to be inflated such that its volume is only partially filled, such that the pressure within the balloon can be maintained at ambient pressure, that is, the pressure within the balloon 33 is approximately equal to the pressure outside of the balloon. This allows the balloon 33 to shape itself anatomically into the vesical trigone 38 and fill the inner urethral opening 39.

In order to allow the filling of the balloon 33, the catheter shaft 32 is provided with an opening 36 or a plurality of such openings in the region covered by the balloon 33. These filler openings 37 do not have to be round and may have, in fact, a square or rectangular shape. It has been shown that this shape prevents this thin film of the balloon from closing the one or more openings.

The balloon 33 is preferably attached in elongated shape to the shaft 32, as described above. The resting volume of the sleeve constructed this way is typically smaller than 0.08 ml, preferably in the range of only 0.02 to 0.04 ml. In many embodiments, the pre-shaped balloon elements may have an operating volume of 5 ml and a wall thickness range of approximately 5 to approximately 10 micrometers. In these specific embodiments with an operating filling volume of 30 ml, the wall thickness of the balloon envelope may preferably be in the range of approximately 5 to approximately 15 micrometers.

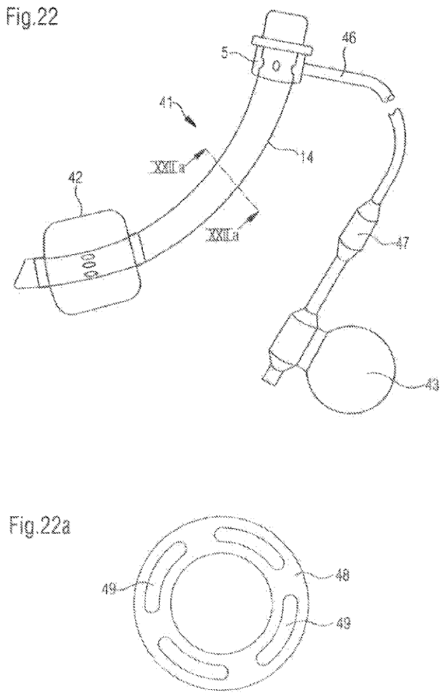

FIG. 22 depicts an exemplary overview of various functionality-optimizing components and describes a tracheal ventilation catheter 41 (tracheal tube) for dynamical tracheal sealing at cyclically alternating thoracic pressures by means of flow-optimized displacement of a pressurized filling medium between a tracheally positioned sealing balloon 42 (cuff) and an extracorporeal control and reservoir element 43.

Here, shape and dimensions of the device according to the invention largely correspond to a traditional tracheal tube. The tracheally sealing balloon is connected with the balloon-supporting catheter shaft at the distal end of the tube, with both tube ends tightly sealed. Preferably, the shaft body 44 comprises a cuff supply line having a large lumen or a plurality of lumens and being integrated into the shaft. At the proximal shaft end 45, the respective supplying lumens are merged and from there connected to the control unit 43 by means of a large-diameter supply line 46. In order to prevent rapid retrograde discharges towards the reservoir and in order to equalize the pressure or volume between the terminal compartments in a delayed fashion, an element 47 with a combined valve and throttle functionality is integrated into the supply line. The communicating volumes of balloon 42, supply line 46, valve throttle element 47, and reservoir or control element 43 together form a common interior space, where a constant pressure defined by the reservoir or controller 43 is maintained. Within the scope of the invention, the preferred medium to fill the communicating interior space is air.

The technology described within the scope of the invention for displacing volumes with optimal speed and minimal resistance and at the same time the lowest possible pressure gradient between a tracheally positioned balloon and an extracorporeal control unit is to allow pressure stabilizing volume compensations within the tracheally sealing balloon that are ideally completed after a maximum of 10 to 20 milliseconds after occurrence of a respiration triggered pressure drop.

FIG. 22a illustrates a cross section of an exemplary shaft profile 48 with multiple lumens. The supply line lumens 49 integrated into the shaft wall are preferably flat or have the smallest possible radial height in order to increase the outer shaft diameter as little as possible.

In the basic design shown in FIGS. 22 and 22a, preferably PVC with a Shore hardness in the range of 80 A to 90 A is used as the material for the shaft 44.

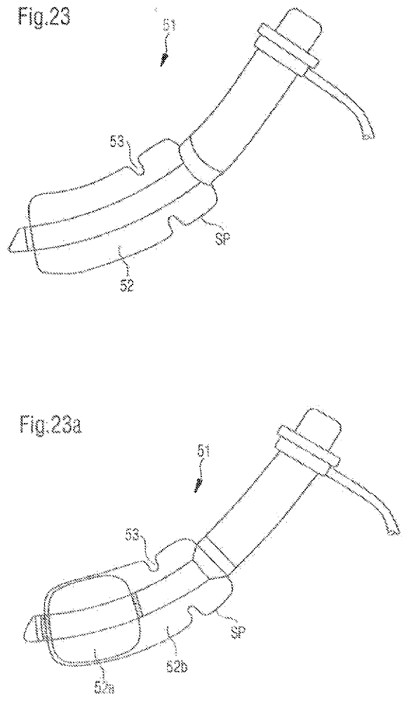

FIG. 23 illustrates a tracheal tube 51 with a balloon element 52 that extends beyond the plane of the vocal folds. The balloon element 52, corresponding to the other embodiments described within the scope of the invention, is preferably made of a molded film material with a residual diameter dimensioned such that it does not need to be stretched in order to seal the tracheal lumen and nestles against the mucous membrane of the organ largely without tension while the excess balloon envelope is folded in. The invention prefers balloon films based on polyurethane that have a wall thickness of preferably 5 to 20 μm, less preferably 20 to 50 μm, in the region of the tracheally sealing balloon segment.

Preferably, polyurethanes with Shore hardnesses 70 A to 95 A or 55 D to 65 D, respectively, are used for the tracheally sealing balloon element according to the invention. More preferably, Shore hardnesses in the range 85 A to 95 A are employed.

While in the simple case the balloon element is dimensioned to seal the region of the transition between the lower and the middle tracheal third, such as is typical for conventional tracheal tubes or tracheal cannulas, the tracheally sealing balloon segment may also be extended in the proximal direction and extend beyond the vocal folds into the region of the supraglottal lower pharynx within the scope of the invention. The body of the balloon element 52 is preferably cylindrical in shape. In the region of the plane of the vocal folds it may be provided with a circular taper 53 for accommodating the vocal folds.

The proximally extended embodiment of the tracheally sealing balloon 52 allows for a particularly large balloon volume that can develop a certain pressure-maintaining buffer effect, when the tracheal cross section in the tracheal portion of the balloon body is enlarged due to respiration or the transmural force onto the tracheally sealing balloon is reduced, respectively. If the proximal balloon end extends beyond the thorax, this extra-thoracic segment is not subjected to thoracic respiratory mechanics, which correspondingly supports the attenuating effect of the extracorporeal volume reserve and further improves the dynamic seal-maintaining functionality of the device according to the invention.

In addition, the large contact area of a proximally extended, tracheally sealing balloon 52 facilitates the largest possible migration path for secretions or agents contained therein.

In FIG. 23a a tracheal tube 51 corresponding to FIG. 23, which comprises two nested cuffs 52a, 52b and which thus, aside from the tracheal seal by means of the inner balloon 52a at relatively higher sealing pressure, also allows tamponading the subglottal space by means of the outer balloon 52b at relatively lower tamponading pressure. Aside from a concentric arrangement, also a sequential, gap-free arrangement of the tracheally sealing cuff (distal) and the subglottal tamponading balloon (proximal) is contemplated.

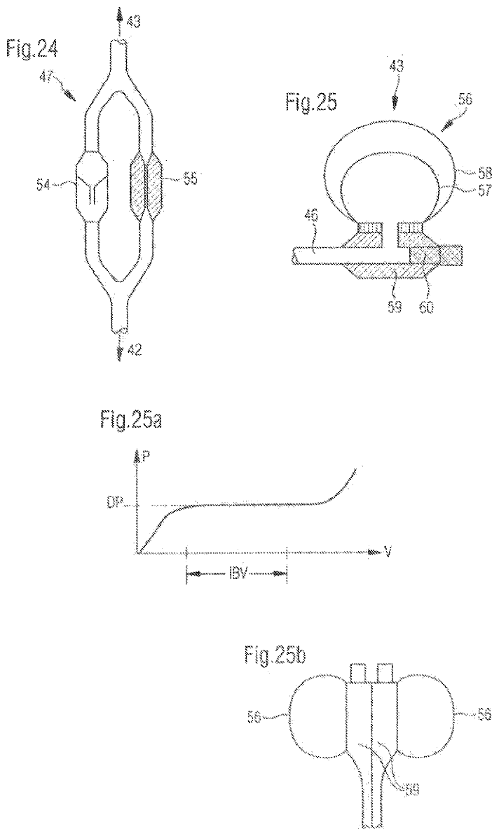

According to FIG. 24, in order to prevent sudden retrograde, potentially sealing-critical discharges of the balloon volume towards the controller or reservoir, such as may, for example, occur when the patient coughs multiple times in short order, the connecting supply line 46 between the shaft 44 and the regulating reservoir unit 43 may be provided with a flow-directing valve 54 that prevents the rapid backflow of filling media. The valve 54 is to be constructed such that it impairs the open, anterograde volume flow from the controller to the balloon as little as possible.

In order to prevent pooling effects of the medium within the balloon 42 caused by the valve 54, the valve 54 is preferably provided with a non-flow-directed bypass valve 55 open to both sides, which allows a slow, delayed pressure or volume equalization between the two terminal compartments, balloon 42 and controller 43. In other words, the throttle 55 is connected in parallel to the check valve 54. The check valve 54 is aligned such that it opens under differential pressure from the reservoir or control unit 43 towards the cuff or sealing balloon 42 and allows the medium to flow rapidly from the reservoir or control unit 43 towards the cuff or sealing balloon 42. If the pressure is inverted, the check valve 54 closes and a flow from the cuff or sealing balloon 42 towards the reservoir or control unit 43 may only occur through the throttle element 55 connected in parallel, which, however, has a smaller open flow cross section, such that the flow per unit time in this flow direction is smaller than that from the reservoir or control unit 43 towards the cuff or sealing balloon 42.

In the simplest embodiment, it is conceivable that the respective sealing valve area is provided with a small bore or opening that allows a correspondingly throttled volume flow.

FIG. 25 illustrates an example embodiment of a combined control and reservoir unit 43. This unit 43 exhibits a few conceptional characteristics of a so-called Lanz controller. As with a Lanz unit, the essential functional component of the unit 43 consists of a special volume-expandable balloon bladder 56 made of highly elastic material, which has a certain pre-formed, for example spherical, base or resting shape 57, which changes into a stretched operating shape 58 when filled. If suitable materials are used, a specific expansion behavior of the balloon bladder may be achieved, where the volume increases when the pressure within the balloon 42 changes in an isobaric fashion. The corresponding, conceptionally desired pressure-volume curve is shown in FIG. 25a. Preferably, the balloon bladder 56 consists of a natural, latex-like material or of a synthetic fabric, such as one related to isoprene. The balloon bladder 56 sits on a base housing 59, in which a one-way valve 60 for filling with air is preferably installed.

FIG. 25a shows an exemplary pressure-volume curve for the reservoir or control unit 43 of FIG. 25, which establishes a constant pressure DP across a specific volume range IBV. Thus, volume may flow out of the expanding balloon 58 towards the balloon 42 without a pressure drop caused by the outflow of volume within the reservoir 56, that is, without leaving the pressure plateau DP. For applications of this control technology for respiratory tubes, the isobaric volume range IBV that can be adjusted this way is to approximately correspond to the freely formed volume of the tracheally sealing balloon 42.

FIG. 25b illustrates a specific double-chambered reservoir balloon arrangement, where two balloon-based reservoirs 56 of FIG. 25 are mounted to a single common base housing 59. One of the chambers facilitates a tracheal sealing pressure of 25 to 30 mbar, whereas the other facilitates the adjustment of a tamponading pressure to 5 to 15 mbar, for example for subglottal tamponading, as described in FIG. 23a.

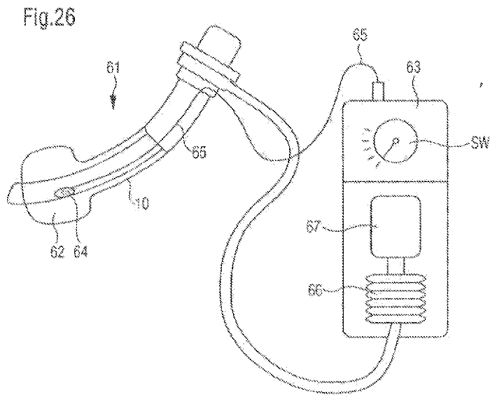

FIG. 26 illustrates a tracheal tube 61 that is provided with a pressure absorbing or pressure sensing element 64 within the interior of the tracheally sealing balloon segment 62. In a preferred embodiment, the pressure sensor 64 is an electronic component which transfers its measurement signal via a wire 65 to an electronically controlled controller 63. The sensing element 64 preferably comprises an absolute pressure sensor. Preferably, sensors on the basis of strain gauges or piezoelectric sensors may be used. The controller 63 has a bellow-like or piston-like reservoir 66, for example, which either displaces volume towards the balloon 62 or removes it from the balloon 62, actuated by means of an actuator 67. The actuator 67 may, for example, be a stepper motor or be constructed as a linear-magnetic actuator. The control of the controller 63 is such that deviations of the filling pressure within the region of the sealing balloon segment 62 are compensated immediately by a corresponding volume displacement or the filling pressure is held constant at a setpoint SW, which may be adjusted at the controller 63. The stabilization of the sealing balloon pressure occurs already at a time, which is optimally prior to the start of the mechanical ventilation stroke or the actual volume flow of respiratory gas into the lung of the patient, respectively. This is particularly relevant for patients who have to expend higher respiratory effort after prolonged controlled mechanical ventilation in order to expand a lung without sufficient volume-expandability to the point that triggers an effective volume flow into the lung. In this phase of isometric tension of the lung within the thorax or the pressure drop within the ribcage accompanying the tension of the stiffened lung, respectively, drops in balloon filling pressure may occur which cause aspiration.

In contrast to a mechanical controller 43 of a simple design, which provides an isobaric reserve volume of preferably 20 to 35 mbar, with the described electronic control unit 63, a pressure may be built up that exceeds the tracheally non-critical sealing pressure of 20 to 35 mbar for a short period of time and may thus sealingly counteract any pressure spikes in the tracheal balloon caused by the patient.

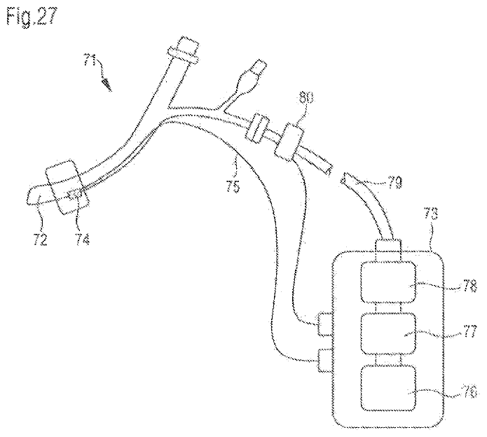

FIG. 27 shows a feedback control system comprising a controller 73 which is connected to a flow-optimized tracheal tube or catheter 71 with a design according to the invention. The catheter 71 comprises an electronic pressure sensor 74 within the sealing balloon 72 which is permanently integrated into the balloon and connected to the controller 73 by means of a wire connection 75. The controller 73 itself consists of a pump module 76 with an optionally integrated reservoir 77 and at least one controlling valve module 78 with an integrated control unit. Setpoints and alarm points may be entered into the controller by the user. Optionally, the controller 73 may also comprise two pump systems, each connected to a reservoir, where the one reservoir holds an overpressure and the other one an underpressure. The gradients or the differential pressure in the controller 73 to the setpoint within the cuff 72, respectively, are controlled independently in an optional embodiment by the controller 73 by means of a learning algorithm, such that the latency to reach the setpoint within the cuff 72 is in the range of 10 to 20 ms. Both the pump functionality and the valve functionality are preferably based on piezoelectric components that may be operated precisely and rapidly as well as in a quiet and energy-saving manner.

Preferably, the inner diameter of the supply line 79 to the extracorporeal connection of the catheter 71 exceeds the diameter of the leg and ideally exceeds it by 30% in order to keep resistance-related flow losses as low as possible.

As an alternative to the pressure sensor 74 integrated into the cuff, a peripheral pressure-converting sensor 80 may be integrated into the supply line and positioned in close proximity to the connector. In this embodiment, a sensor 74 integrated into the cuff 72 may be omitted, whereby a certain latency in the control time is accepted.

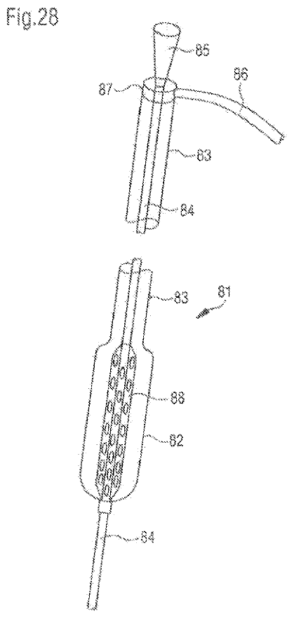

FIG. 28 illustrates a trans-esophageal probe 81 for supplying or draining substances or media into or through the stomach, the probe being provided with an esophageally sealing balloon element 82. The proximal balloon end 83 may be extended into the extracorporeal connector 85. There, it may be connected in a tightly sealing fashion to the sealing connector element 87 that is freely moveable on the shaft 84, which element transitions in turn into a supply line 86 with a large lumen, which is connected to a control mechanism 43 according to the invention. Under an esophageal seal, the control component 43 sets a sealing pressure plateau DP of approximately 20 to 30 mbar within the communicating interior space.

For improved stationary retention of the tamponading sealing balloon segment in the esophagus, the segment may be provided with a non-collapsible profile 88 in the esophageal region, which, in the case of a peristaltic contraction of the esophagus, discharges a volume from the balloon segments prior to the contraction wave through or below the profile into regions that have already been released by the wave. This prevents the mushrooming of filling medium prior to the contraction wave, which would lead to a transport of the entire device towards the stomach. Corresponding profiles are already described in EP 0929339 B1 and may be used in the fill scope disclosed therein within the invention.

List of reference numerals

| | | | |
|---|---|---|---|
| 1 | balloon tube film | 21 | PVDC layer |
| 2 | balloon portion | 22 | PVC layer |
| 2a | distal balloon region | 23 | invagination |
| 2b | central balloon region | 24 | web-like element |
| 3 | shaft tube portion | 24a | wall layer |
| 3a | intrarectal portion | 24b | wall layer |
| 3b | transanal portion | 25 | eyelet-like formation |
| 3c | preanal portion | 26 | entry region |
| 4 | compartment | 27 | channel-like formation |
| 5 | head unit | 28 | end-face side |
| 6 | tube portion, lumen | 29 | graph |
| 7 | wall profile | 30 | graph |
| 8 | U-shaped extension | 31 | urinary catheter |
| 9 | U-shaped extension | 32 | shaft |
| 10 | barrier layer | 33 | balloon |
| 11 | polyurethane layer | 34 | opening |
| 12 | polyamide layer | 35 | strip |
| 13 | polyvinyl chloride layer | 36 | strip |
| 14 | outer PUR layer | 37 | opening |
| 15 | inner PUR layer | 38 | vesical trigone |
| 16 | barrier layer | 39 | inner urethral opening |
| 17 | inflection point | 41 | tracheal tube |
| 18 | balloon shaft end | 42 | sealing balloon |
| 19 | TPE layer | 43 | reservoir, control unit |
| 20 | promoter layer | 44 | shaft body |
| 45 | shaft end | 72 | sealing balloon |
| 46 | supply line | 73 | controller |
| 47 | valve throttle element | 74 | pressure sensor |
| 48 | shaft profile | 75 | wire connection |
| 49 | supply line lumen | 76 | pump module |
| 51 | tracheal tube | 77 | reservoir |
| 52 | balloon element | 78 | valve module |
| 52a | cuff | 79 | supply line |
| 52b | cuff | 80 | pressure sensor |
| 53 | circular taper | 81 | trans-esophageal probe |
| 54 | valve | 82 | balloon element |
| 55 | throttle | 83 | proximal balloon end |
| 56 | balloon bladder | 84 | shaft |
| 57 | resting shape | 85 | connector |
| 58 | operating shape | 86 | supply line |
| 59 | base housing | 87 | connector element |
| 60 | one-way valve | 88 | non-collapsible profile |
| 61 | tracheal tube | D | distal balloon end |
| 62 | balloon segment | GR | large radius |
| 63 | controller | P | proximal balloon end |
| 64 | sensing element | R | plane |
| 65 | wire | | |
| 66 | reservoir | | |
| 67 | actuator | | |
| 71 | tracheal tube | | |

1. A device for supplying and/or draining substances in a minimally irritating, tissue-compatible manner that is preferably adapted to the movements of the body and its organs, comprising a balloon (2), which may be placed in an interior space of the body and filled from outside the body, which balloon—optionally together with a supplying and/or draining tube or shaft segment (3, 3a) that carries the balloon (2)—surrounds a fillable compartment (4), to which a tube or shaft-like segment (3b) is attached that connects the interior space to the body surface, wherein the balloon (2) is produced by blow molding from a tube blank made of a multi-layer film-like material, characterized in that the tube blank is extruded in multiple layers, wherein, in addition to at least one elastically deformable layer of polyurethane (PUR), at least one not elastically deformable, odor- and/or media-tight barrier layer of ethylene vinyl alcohol copolymer (EVOH) or of polyvinylidene chloride (PVCD) or of polyamide (PA) or of a thermoplastic polyamide elastomer (TPE-A) is provided in the multi-layer material, and wherein the tube blank that has undergone blow molding has been extruded in a previous separate method step, such that the for the blow molding process necessary ratio of crystalline to amorphous material proportions is established in the layer of polyurethane.

2. The device according to claim 1, characterized in that the total thickness of all elastically deformable layers corresponds to at least 1.5 times the total thickness of all not elastically deformable, odor- and/or media-tight barrier layers.

3. The device according to claim 1, characterized in that the balloon (2) is made by means of a blow molding process based on thermal forming, in particular, by applying blowing pressure onto a tube blank having a relatively small lumen and thick walls and previously (co) extruded in a separate manufacturing step in order to expand said blank to a relatively larger operating diameter, thereby forming it into a relatively thin-walled film tube, in particular, by nestling the heated tube blank into a gradually or incrementally heated, profiled mold wall and shaping it at high temperature while resting against the tempered mold wall.

4. The device according to claim 1, characterized in that the connection of the plurality of layers (2, 3, 10) of the balloon envelope (13) is manufactured by coextrusion of the different layers (2, 3, 10) without using an adhesion promoter between the respective film layers.

5. The device according to claim 1, characterized in that the maximal radial stretch ratio does not exceed 1:8 (stretched tube blank fixed within the mold at the start of the molding of the balloon under blowing pressure:largest molded diameter) to ensure the structural continuity and integrity of the barrier layers, respectively, in the balloon blow molded from the tube blank by means of thermal forming where the tube blank is maximally stretched 1.5 times during blow molding, and that more preferably a maximal radial stretch ratio does not exceed 1:5.

6. The device according to claim 1, characterized in that the at least one PUR layer (2) is made of thermoplastic PUR of a type having a water absorption of 5% or less according to DIN ISO 62, preferably having a water absorption of 2% or less according to DIN ISO 62.

7. The device according to claim 1, characterized in that, in addition to or instead of an elastic polyurethane layer, also inelastic material layers are provided in the multi-layered material, for example material layers based on PVC and PE.

8. The device according to claim 1, characterized in that layers having elastic deformation characteristics, such as those preferably provided by thermoplastic polyurethanes (TPU), are proportionally predominant in the multi-layered material, for example thermoplastic, ester- and ether-based polyurethanes, preferably having a Shore hardness in the range of 80 A to 95 A as well as in the range of 55 D to 70 D or in the range of 55 D to 65 D.

9. The device according to claim 1, characterized in that a layer (2) made of elastically deforming PUR material is combined with a layer (3) made of plastically deforming material, preferably made of plastically deformable PVC.

10. The device according to claim 1, characterized in that one or more odor-tight or media-tight barrier layers are integrated within the multi-layered material.

11. The device according to claim 1, characterized in that the layer (2), instead of being made of thermoplastic PUR of a type having a water absorption of 5% or less according to DIN ISO 62, preferably having a water absorption of 2% or less according to DIN ISO 62, is made of a material having comparable elastic properties, preferably of an organic material having comparable elastic properties.

12. The device according to claim 1, characterized in that the total wall thickness of the balloon envelope is equal to or smaller than 50 μm, preferably equal to or smaller than 40 μm, more preferably equal to or smaller than 30 μm.

13. The device according to claim 1, characterized in that the ratio of the wall thickness proportion of the at least one PUR layer (2) to the wall thickness proportion of the at least one layer (3) of a non-elastic material, for example PVC, is between 1:1 and 1:5, preferably between 1:2 and 1:4, more preferably around 1:3.

14. The device according to claim 1, characterized in that by combining at least one EVOH or PVDC layer (3) with at least one PUR layer (2) the migration of fluids, for example of air or polar liquids, in particular of water, through the wall (1) of the balloon envelope (13) is reduced.

15. The device according to claim 1, characterized in that the tube blank is made of a three-layered or multi-layered material.

16. The device according to claim 1, characterized by a three-layered balloon envelope.

17. The device according to claim 1, characterized in that a gas- and/or water vapor-tight barrier layer (10), preferably made of PVDC or EVOH, is arranged between the elastically deformable PUR layer (2) and the not elastically deformable layer (3), for example made of PVC.

18. The device according to claim 17, characterized in that the wall thickness proportion of the not elastically deformable layer (3), for example made of PVC, is larger than the wall thickness proportions of the elastically deformable PUR layer (2) and/or the gas- and/or water vapor-tight barrier layer (10), preferably made of PVDC or EVOH.

19. The device according to claim 17, characterized in that the ratio between the wall thickness proportion of the gas- and/or water vapor-tight barrier layer (10), preferably made of PVDC or EVOH, and the wall thickness proportion of the not elastically deformable layer (3), for example PVC, is between 1:1 and 1:5, preferably between 1:2 and 1:4, more preferably around 1:3.

20. The device according to claim 1, characterized in that during the in situ placement of a residually formed balloon body, that is, a balloon body that is formed with excess balloon material along the balloon circumference, typical, into the balloon interior invaginated infoldings (8) of the excess residual balloon envelope are formed.

21. The device according to claim 1, characterized in that the supplying and/or draining extracorporeal tube segment connected to the head unit has such a thin wall thickness that it folds in a radially inward direction or collapses into a flat, strip-like structure when an external force is applied, and when the applied external force diminishes, it spontaneously straightens in an elastic manner until it reaches an at least partially open, partially rounded cross section.

22. The device according to claim 1, characterized in that the wall of the supplying and/or draining tube structure (6) of the transluminal segment (3b) of the device that forms the access path to the interior space allows the transluminal segment (3b) to transition into the state of an elastically operative, radially directed folding or invagination with reduced cross section when a corresponding force is applied by the abutting structures of the body, and to straighten into its original shape from the deformed state with reduced cross section, when the external forces onto the transluminal segment (3b) diminish or the respective lumen leading to the interior space opens, respectively.

23. The device according to claim 1, characterized in that infoldings (8) that are invaginated into the balloon interior have turned-over formations (6) with an eyelet-like cross section, which preferably extend or continue as channel-like formations (9) in the longitudinal direction of the balloon (13), that is, between the distal and proximal end-face sides (9) of the balloon.

24. The device according to claim 23, characterized in that the turned-over formations (6) with an eyelet-like cross section, which preferably extend or continue as channel-like formations (9) in the longitudinal direction of the balloon (13), have an opening diameter between 30 μm and 120 μm, preferably an eyelet diameter between 40 μm and 80 μm, at a filling pressure of the balloon (13) of 30 mbar.

25. The device according to claim 23, characterized in that as a result of the combination with at least one layer (3) made of a non-elastic material, for example of PVC, the opening diameter of the eyelet-like or channel-like formation (6, 9) is reduced compared to the opening diameter of an eyelet-like or channel-like formation (6, 9) with a pure PUR layer (2) of the same type of material and the same layer thickness.

26. The device according to claim 23, characterized in that at least one layer (3) made of a non-elastic material, for example made of PVC, is of a plastic, non-elastic nature, such that its flat deformation, bending or twisting has an attenuating effect on the opening kinetics of eyelet- or channel-like formations (8) when the filling pressure of the balloon (13) changes in situ.

27. The device according to claim 23, characterized in that the elastically caused opening and/or expansion of the eyelets and/or channels is slowed as a result of the combination of at least one PUR layer (2) with at least one layer (3) made of a non-elastic material, for example of PVC, when the pressure is reduced intermittently or cyclically within the balloon.

28. The device according to claim 23, characterized in that, as a result of the combination of at least one PUR layer (2) with at least one layer (3) made of a non-elastic material, for example of PVC, the elastic straightening effect is reduced in the region of the eyelet-like or channel-like turned-over formations (6, 9) such that the cross-sectional areas of the secretion-conveying, eyelet- and channel-like structures (6, 9) are both reduced in size and minimized in case of cyclical variations of the balloon filling pressure, in contrast to a single-layered elastic balloon film (2) made of only PUR.

29. The device according to claim 23, characterized in that the eyelet- or channel-like turned-over formations (6, 9) retain their sealing properties against fluids, in particular against liquids, as long as at least one of the balloon filling pressure and the lower limits of the permissible variations of the balloon filling pressure remains at or above 5 mbar.

30. The device according to claim 23, characterized in that the cross-sectional areas of the eyelet- or channel-like turned-over formations (6, 9) do not increase by more than 25%, preferably only by 20% or less, as long as at least one of the balloon filling pressure and the lower limits of the permissible variations of the balloon filling pressure remains at or above 5 mbar.

31. The device according to claim 23, characterized in that the cross-sectional areas of the eyelet- or channel-like turned-over formations (6, 9) do not increase by more than 25%, preferably only by 20% or less, as long as at least one of the pressure amplitude and the difference between the two extremes of the balloon filling pressure remains in a range between 5 mbar and 30 mbar.

32. The device according to claim 1, characterized in that a corrugated, annular or helical-like profile shape (7) of the wall of the supplying and/or draining tube structure (3, 3a) supporting the balloon (2) or balloon portion (2a) retained within the respective interior space or organ allows the supplying and/or draining tube structure (3, 3a) to radially fold, with reduced cross section, the portion (3, 3a) of the device positioned within the interior space during insertion and counteract the filling pressure within the retaining balloon (2) without reducing the supplying and/or draining cross section by means of its specific straightening effect and promptly straighten itself from the radially deformed state back into the initial shape specified during production, thereby keeping the respective mouth of the supplying and/or draining lumen (6) open towards the interior space.

33. The device according to claim 32, characterized in that the corrugated, annular or helical profile shape (7) of the profile is manufactured by means of a single-layered or multi-layered immersion process or by means of an injection molding process.

34. The device according to claim 1, characterized in that it is suitable for insertion into the trachea, the esophagus, the urethra or the intestine.

35. A method for producing a device for supplying and/or draining substances in a minimally irritating, tissue-compatible manner that is preferably adapted to the movements of the body and its organs, the device comprising a balloon (2), which may be placed in an interior space of the body and filled from outside the body, which balloon—optionally together with a supplying and/or draining tube or shaft segment (3, 3a) that carries the balloon (2)—surrounds a fillable compartment (4), to which a tube or shaft-like segment (3b) is attached that connects the interior space to the body surface, wherein the balloon (2) is produced by blow molding from a tube blank made of a multi-layer film-like material, characterized in that a tube blank having multiple layers is manufactured by multi-layered extrusion, wherein, in addition to at least one elastically deformable layer of polyurethane (PUR), at least one odor- and/or media-tight barrier layer of ethylene vinyl alcohol copolymer (EVOH) or of polyvinylidene chloride (PVCD) or of polyamide (PA) or of a thermoplastic polyamide elastomer (TPE-A) is provided in the multi-layer material, and in that the multi-layered tube blank is subsequently at least partially molded into a balloon by thermal forming in a blow molding process.

36. The method according to claim 35, characterized in that the coextruded tube blank with relatively small lumen and thick walls is stretched in its longitudinal direction during the thermal forming and preferably fixed in the stretched state.

37. The method according to claim 36, characterized in that the coextruded tube blank with relatively small lumen and thick walls, preferably in the stretched state, is expanded to a relatively larger operating diameter by applying blowing pressure and thereby transformed into a relatively thin-walled film tube, wherein the tube is nestled into a gradually or incrementally heated, profiled mold wall and finally shaped at high temperature while resting against the completely tempered mold wall.

38. The method according to claim 35, characterized in that the tube blank is manufactured by means of three- or multi-layered film extrusion.

39. The method according to claim 35, characterized in that at least one barrier layer, for example made of EVOH or PVDC or PA or TPE-A, is coextruded with at least one support layer, for example made of TPU, PUR or PVC or PE.

40. The method according to claim 35, characterized in that the multi-layered extrusion occurs by simultaneously supplying a plurality of different material melts to a single, common extrusion head.
